(19) United States
(12) Patent Application Publication
(10) Pub. No.: US 2012/0225087 A1
(43) Pub. Date: Sep. 6, 2012

(54) NICOTINE HAPTENS, IMMUNOCONJUGATES AND THEIR USES

(75) Inventor: Kim D. Janda, San Diego, CA (US)

(73) Assignee: The Scripps Research Institute, LaJolla, CA (US)

(21) Appl. No.: 13/261,217

(22) PCT Filed: Sep. 14, 2010
(86) PCT No.: PCT/US2010/002489

§ 371 Date: May 18, 2012

Related U.S. Application Data
(60) Provisional application No. 61/276,679, filed on Sep. 14, 2009.

(51) Int. Cl. A61K 39/385; C12N 5/078; C07K 14/00; C07K 16/44; A61K 39/395; C07D 401/04; A61P 37/04

(52) U.S. Cl. 424/175.1; 546/276.4; 435/325; 424/184.1; 530/389.8; 530/358; 530/363; 530/405

(57) ABSTRACT

The present invention provides novel nicotine hapten compounds and nicotine immunoconjugates which can be used for in vivo production of antibodies that specifically bind to nicotine. The invention also provides methods of using vaccines comprising the nicotine immunoconjugates in active or passive immunization protocols. The compositions and methods of the invention are useful for prevention and treatment of nicotine addiction.

### CROSS-REFERENCE TO RELATED APPLICATIONS

The subject patent application claims the benefit of priority to U.S. Provisional Patent Application No. 61/276,679 (filed Sep. 14, 2009). The full disclosure of the priority application is incorporated herein by reference in its entirety and for all purposes.

### COPYRIGHT NOTIFICATION

Pursuant to 37 C.F.R. §1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Nicotine, (S)-(−)-1-methyl-2-(3-pyridyl)pyrrolidine, is an addictive substance that is richly present in cigarettes, cigars, pipes and smokeless tobacco. Smoking of cigarettes, cigars, and pipes is a prevalent problem in the United States and worldwide. Nicotine targets the mesolimbic dopamine system and binds to nicotinic cholinergic receptors resulting in physiological dependence. The psychopharmacological effects of nicotine in dependent tobacco smokers include tranquilization, weight loss, decreased irritability, reduction in craving for cigarettes, increased alertness, and improved cognitive functioning. Deprivation of nicotine results in withdrawal symptoms and an increase in nicotine-seeking behavior.

A number of therapies are currently available for treating and preventing nicotine addiction. These treatment approaches, which usually depend solely on unaided compliance or on the administration of nicotine itself for rehabilitation, are largely ineffective. For example, the two most common therapies, nicotine transdermal patch and nicotine chewing gum, have afforded inadequate long-term success rates of less than <20%. Other problems or side effects are also known to be associated with these therapies. In particular, there is low penetration of nicotine into the bloodstream and therefore an increased desire to smoke. Problems such as mouth irritation, jaw soreness, nausea, have been associated with use of nicotine chewing gum. Problems such as skin irritations, sleep disturbance, and nervousness have been associated with use of nicotine transdermal patches.

There is a need in the art for better means for treating nicotine addiction. The present invention addresses this and other unfulfilled needs in the art.

### SUMMARY OF THE INVENTION

In one aspect, the invention provides a hapten compound of formula (I):

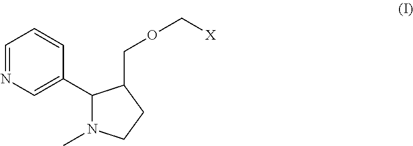

wherein X is a linker moiety that does not contain a thiol group.

In another aspect, the invention provides an immunoconjugate of formula (II):

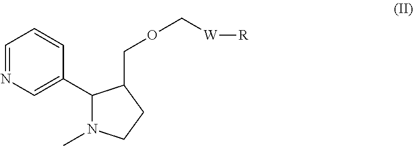

wherein W is a linker moiety that is covalently linked to a carrier moiety R, and wherein the covalent linkage is not a thioether bond. Also provided in the invention is a pharmaceutical composition that comprises an immunologically effective amount of the immunoconjugate and a physiologically acceptable vehicle. The pharmaceutical composition can further comprise an appropriate adjuvant.

In a related aspect, the invention provides a method of inducing an anti-nicotine immune response in a subject. The method entails immunizing the subject with an immunologically effective amount of the immunoconjugate or pharmaceutical composition disclosed herein.

In another aspect, the invention provides a method of preparing an immunoconjugate of formula III:

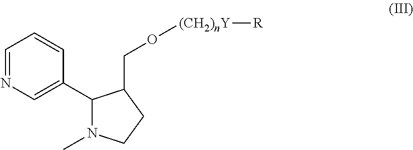

wherein Y is a functional group that facilitates linkage to a carrier moiety, R is a carrier moiety and n is an integer from about 3 to about 8. The method involves first converting compound A:

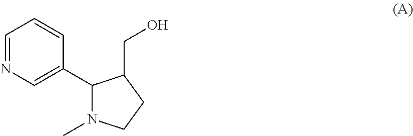

to compound B:

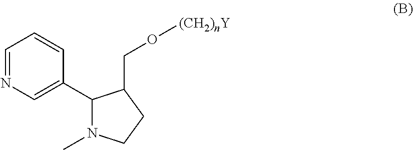

It is then followed by converting compound B to the immunoconjugate of formula III.

In yet another aspect, the invention provides an antibody that binds to the immunoconjugate disclosed herein. Further provided is a pharmaceutical composition that comprises the antibody and a physiologically acceptable carrier or vehicle.

A further understanding of the nature and advantages of the present invention may be realized by reference to the remaining portions of the specification and claims.

### BRIEF DESCRIPTION OF THE DRAWINGS

FIG. 1 shows the scheme of synthesis of nicotine hapten AM1 and generation of appropriate hapten-protein conjugates.

FIG. 2 shows average NIC-BSA titer of n=5 rats elicited during course of self-administration. *p<0.05.

FIG. 3 shows average number of infusions taken under an FR1-TO-20 s schedule of reinforcement by vaccinated and control groups. #p≦0.10; *p<0.05.

### DETAILED DESCRIPTION OF THE INVENTION

### I. Overview

The present invention is predicated in part on a new class of nicotine haptens and hapten-carrier immunoconjugates generated by the present inventors which are useful in immunopharmacotherapy for the treatment of nicotine addiction. Immunopharmacotherapy aims to use highly specific antibodies to blunt passage of drug into the brain thus minimizing reinforcing effects on the reward pathways of the central nervous system. Nicotine and its metabolite cotinine are small molecular weight molecules, and need to be appended to macromolecules in order to elicit an immune response. As both of these structures do not possess suitable functional groups for these purposes, the target scaffolds must be functionalized with an appropriate linker. Linker-nicotine regiochemical attachment has proven to be crucial for proper immune stimulation both in terms of the amount of antibody elicited as well as obtaining the desired antibody specificity. For nicotine, several linker attachment sites have been investigated and of particular note is that of Langone et al. (Biochemistry 12:5025-5030, 1973). In the original report, trans-3′-succinylmethylnicotine was generated and coupled to different macromolecules. Immunization of these conjugates into albino rabbits in formulation with complete Freund's adjuvant generated antibodies which allowed detection of picomolar levels of nicotine in various tissues and biological fluids even in the presence of cotinine without detectable antibody cross-reactivity. Since the original Langone report, a plethora of haptens of the same general structure, i.e., functionalized at the 3′ position, have become the most widely prepared and studied molecules of all nicotine haptens (e.g., Hieda et al., Int J Immunopharmacol 22:809-819, 2000). However, there are various problems associated with the nicotine haptens that have been reported in the art, e.g., poor hapten stability, additional immunogenic moiety other than the nicotine target structure, and high variation of antibody titers. For example, the range of antibody titers obtained in Hieda et al. (Int J Immunopharmacol 22:809-819, 2000) is highly variable up to a full order of magnitude. This high variation of titer is significant as success in promoting nicotine cessation is directly related to the amount of circulating antibodies and thus high titer variability directly translates to high abstinence rate variability.

The present inventors designed and synthesized a class of nicotine derivative compounds which possess advantageous properties over nicotine haptens known in the art. Hapten-protein immunoconjugates based on the hapten compounds of the invention confer hapten stability, and are also effective in generating antibodies in animals with satisfactory titers as well as affinity and specificity for nicotine. In addition, vaccination of animals with nicotine dependence with the immunoconjugates is able to successfully induce certain behavioral changes that suggest efficacy of the immunoconjugates to aid nicotine cessation. Specifically, as detailed in the Examples below, the immunoconjugates of the invention were able to elicit elevated levels of antinicotine antibodies in both mice and rat rodent models. The native antigenicity of the hapten is highlighted by the fact that high antibody titer levels were obtained regardless of carrier protein when checked for cross reactivity with a non-immunized nicotine analogue (NIC). In addition, it was shown that vaccination with the immunoconjugates allowed for generation of nicotine specific antibodies even with concurrent self-administration of high doses of naïve drug in rats. This result suggests that vaccination can be initiated before smoking cessation begins, even in heavy smokers, without affecting vaccine immunogenicity. Furthermore, the presence of these anti-nicotine antibodies actively altered the intravenous self-administration pattern in immunized subjects, where protective effects of vaccination are mirrored in an increased drug intake.

In accordance with these discoveries, the invention provides novel nicotine hapten compounds, and immunoconjugates comprising the haptens. The invention also provides methods of producing such immunoconjugates and therapeutic methods of using the immunoconjugates to treat subjects with nicotine dependence or addiction.

The following sections provide more detailed guidance for practicing the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

As used herein, the term “adjuvant” refers to immunological agents that may stimulate the immune system of a subject and increase the response to a vaccine, without having any specific antigenic effect in itself. It encompasses any substance that acts to accelerate, prolong, or enhance antigen-specific immune responses when used in combination with specific vaccine antigens. Thus, an adjuvant suitable for the present invention is capable of enhancing the immune response against the immunoconjugates described herein.

As used herein and in the art, the term “hapten” refers to a small molecule which elicits a detectable immune response when attached to a carrier moiety. When constructed as an immunoconjugate, the hapten is characterized as the specificity-determining portion of the immunoconjugate. Antibodies generated in response to immunization with immunoconjugates of the invention are also capable of reacting with the hapten or with nicotine in its free state, and are thus also useful in a variety of assays.

“Active immunization” refers to the induction of an immune response in a subject by providing an antigen, for example, an immunoconjugate. Immunoconjugates are suitably included in a pharmaceutical composition containing a physiologically acceptable vehicle or carrier such that an immunologically effective amount of the immunoconjugate can be delivered to a subject.

A “carrier moiety,” as used herein, refers to a conjugation partner capable of enhancing the immunogenicity of the hapten. Carrier moieties are well known in the art and are generally proteins.

An “immunologically effective amount” means an amount of an immunogen (e.g., an immunoconjugate disclosed herein) which is capable of inducing an immune response against the immunogen and/or generating antibodies specific for the immunogen or other agents which share immunological features of the immunogen of interest, e.g., nicotine.

“Passive immunization” refers to short-term immunization achieved by the transfer of antibodies to a subject.

A “physiologically acceptable” vehicle is any vehicle or carrier that is suitable for in vivo administration (e.g., oral, transdermal, intramuscular, or parenteral administration) or in vitro use, i.e. cell culture.

The term “subject” refers to a vertebrate, suitably a mammal, more suitably a human.

Vaccine refers to a biological preparation that, when administered to a subject, elicits an immune response (including production of specific antibodies) against an agent (e.g., nicotine) or that improves immunity to a particular disease. A vaccine typically contains a small amount of an immunogen (e.g., a nicotine derivative) that immunologically resembles the agent of interest or a microorganism. The immunogen stimulates the body's immune system to recognize the agent as foreign, destroy it, and “remember” it, so that the immune system can more easily recognize and destroy the agent that it later encounters.

### III. Design and Synthesis of Nicotine Haptens and Immunoconjugates

Because nicotine is inherently non-immunogenic, the inventors have designed compounds that have structural and stereochemical features of nicotine such that antibodies to these compounds will cross-react with nicotine. Haptens in accordance with the present invention may be synthesized de novo or from a nicotine-related compound. In some embodiments, nicotine or a nicotine derivative compound is employed as the starting material in synthesis of the haptens. In other embodiments, the nicotine haptens can be generated by de novo synthesis in accordance with standard chemical methods well known in the art. The haptens of the present invention can be coupled with a carrier protein so that they can elicit an enhanced immune response in a subject. The immune response includes the production of hapten-specific antibodies which can cross-react with nicotine.

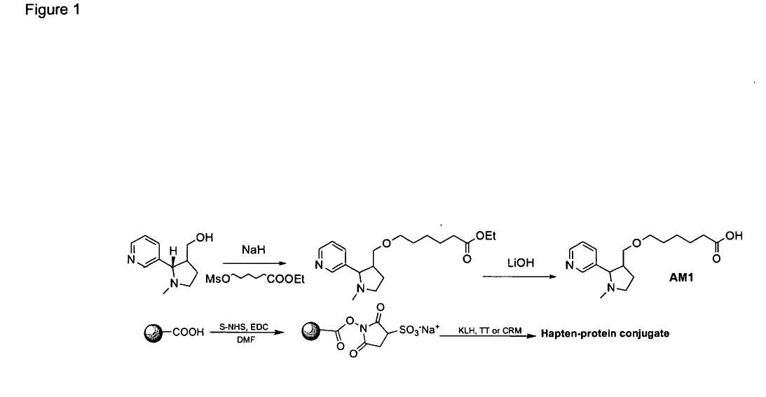

Some haptens in accordance with the invention have the structure shown in formula (I):

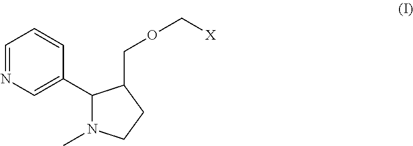

wherein X is a linker moiety. Haptens of formula I may be synthetically derived to mimic the molecular features of nicotine. As noted above, the hapten may be synthesized with or without the use of nicotine or nicotine derivatives as a reactant in the synthesis process. An exemplary method of producing the hapten of formula (I) is described in the Examples below. An important aspect of the hapten structure is the use of a simple ether linkage as opposed to an amide moiety commonly used in nicotine hapten designs. As demonstrated in the Examples herein, the ether appendage not only provides hapten stability but also allows for a “masked” appendage site which focuses the immune response onto the desired nicotinic target. Haptens with such structural design are able to generate effective anti-nicotine antibodies in vivo. A specific example of the nicotine haptens of the present invention, designated AM1, is shown in FIG. 1.

The haptens of the invention as described above can be linked to a carrier moiety to generate nicotine immunoconjugates. The immunoconjugates can be readily produced using standard methods known in the art. To generate the immunoconjugates, the nicotine hapten can be covalently or non-covalently conjugated to the carrier moiety. In some embodiments, the nicotine hapten is conjugated to the carrier moiety via a linkage that is not a thioether bond. In some embodiments, the linker moiety X is conjugated to the carrier moiety via a covalent bond. Depending on the functional group in the linker moiety X and the carrier moiety, various covalent bonds can be used to conjugate the nicotine hapten to the carrier moiety. In some embodiments, the linker moiety is first activated to generate a functional group that can readily react with an amino acid residue in the carrier moiety to form a covalent linkage. Specific examples of immunoconjugates thus formed are exemplified in the Examples below. In some other embodiments, the carrier moiety can be modified with a derivatizing molecule or spacer molecule in order to generate a functional group for reacting with the nicotine hapten. Derivatizing molecules suitable for practicing the present invention are well-known in the art.

Various carrier moieties can be employed to produce the immunoconjugates of the present invention. In some preferred embodiments, the carrier moiety is a protein. For instance, proteins derived from bacteria or viruses, such as tetanus toxoid (TT), diphtheria toxoid or related protein such as diphtheria toxin cross-reactive mutant 197 (CRM), cholera toxoid, members of the LTB family of bacterial toxins, retrovirus nucleoprotein (retro NP), rabies ribonucleoprotein (rabies RNP), vesicular stomatitis virus nucleocapsid protein (VSV-N), recombinant pox virus subunits, and the like may be used. Other suitable carrier moieties include keyhole hemocyanin (KLH), edestin, thyroglobulin, bovine serum albumin, human serum albumin, red blood cells such as sheep erythrocytes, (SRBC), as well as polyamino acids such as poly(D)lysine, poly(D)glutamic acid and the like. Polymers also can be used, e.g., carbohydrates such as dextran, mannose, or mannan.

There are a wide range of available methods for linking a hapten to a carrier moiety, any of which are suitably adapted for use in the present invention. As discussed above, some of the nicotine haptens of the invention contain a simple ether group which is connected to a linker moiety X. The linker moiety may be monovalent or divalent depending on whether the carrier moiety is covalently attached to the linker moiety. In some embodiments, the linker moiety does not contain a thiol group. In some embodiments, the linker moiety is an activated acyl. The length and nature of the linker moiety is such that the hapten is displaced a sufficient distance from the carrier moiety to elicit a suitable antibody response to the hapten in vivo. Suitable linker moieties include:
—OY,
—OCH₃,
—OCO(CH₂)ₙCOY,
—OCO(CH₂)ₙCNY,
—OCO(CH₂)ₙY,
—OCOCH═Y,
—OCOCH(O)CH₂,
—OCOCH(OH)CH₂Y,
—OCO(CH₂)ₙCH(OH)CH₂Y,
—OCO(CH₂)ₙCH(O)CH₂Y,
—OCOC₆H₅,
—O(CH₂)ₙY
—CO₂Y,
—COY,
—CO(CH₂)ₙCOY,
—CO(CH₂)ₙCNY,
—CONH(CH₂)ₙY,
—CH₂OCO(CH₂)ₙCOY,
—CH₂OCO(CH₂)ₙCNY,
—(CH₂)ₙY,
—CH₂Z(CH₂)ₙY,
—(CH₂)ₙ—C₆H₁₀—(CH₂)ₘ—COY
—(CH₂)ₙ—C₆H₄—(CH₂)ₘ—COY
—NH(CH₂)ₙCOY
—NH(CH₂)ₖC₆H₁₀—(CH₂)ₘCOY
—NH(CH₂)ₘC₆H₄—(CH₂)ₚCOY
—NHCO(CH₂)ₙCOY
—NHCO(CH₂)ₖC₆H₁₀—(CH₂)ₘCOY
—NHCO(CH₂)ₘC₆H₄—(CH₂)ₚCOY
—C≡C—(CH₂)ₙNHY
—C≡C—(CH₂)ₙCOY
—C≡C—(CH₂)ₙC₆H₁₀—(CH₂)ₘCOY
—C≡C—(CH₂)ₙC₆H₁₀—(CH₂)ₘNHY
—C≡C—(CH₂)ₘC₆H₄—(CH₂)ₚCOY
—C≡C—(CH₂)ₘC₆H₄—(CH₂)ₚNHY
—CH═CH—(CH₂)ₙNHY
—CH═CH—(CH₂)ₙCOY
—CH═CH—(CH₂)ₙC₆H₁₀—(CH₂)ₘCOY
—CH═CH—(CH₂)ₙC₆H₁₀—(CH₂)ₘNHY
—CH═CH—CH₂)ₘC₆H₄—(CH₂)ₚCOY
—CH═CH—(CH₂)ₘC₆H₄—(CH₂)ₚNHY
—SCO(CH₂)ₙCOY
—SCH₂(CH₂)ₙY
—(CH₂)ₙ—R₁—(CH₂)ᵣ—R₂—Y
—Z(CH₂)ₙY
—ZCO(CH₂)ₙCOY
   wherein n is an integer from about 0 to about 20, or in some embodiments from about 1 to about 12, from about 2 to about 10, or about 3 to about 6; m is an integer from about 0 to about 6; k is an integer from about 0 to about 20; p is an integer from about 0 to about 6; r is an integer from about 1 to about 20; Z is selected from the group consisting of —O—, —CH₂—, and —NH—; R₁ and R₂ are independently selected from the group consisting of —NHCO—, —CONH—, —CONHNH—, —NHNHCO—, —NHCONH—, —CONHNHCO—, and —S—S—; and Y is selected from the group consisting of —H, —OH, ═CH₂, —CH₃, —OCH₃, —COOH, halogen, acyl, activated acyls, such as 2-nitro-4-sulfobenzoate and N-oxysuccinimidate, alkyl, N-maleimides, imino acylate, isocyanates, isothiocyanates, haloformate, vinylsulfone, imidoester, phenylglyoxalate, hydrazide, alkynyl, azido, amino, N-hydroxysuccinimidate, —O—, —(CH₂)ₘ—, —S—S—, —NH—, —C(O)O—, —C(O)NH—, —N═N—, —N═N═N—, —CH═CH— and —C≡C—. Other suitable linkers of sufficient length and flexibility may also be used with the present invention. A wide range of reagents and/or active groups may be used to facilitate cross-linking of a hapten to a carrier moiety.

The carrier moiety may be modified by methods known to those skilled in the art to facilitate conjugation to the hapten, e.g., by succinylation. About 1 to about 100 haptens may be conjugated to a carrier moiety, more preferably 1-70, 1-50, or 1-25 haptens may coupled to the carrier moiety.

In a related aspect, the invention provides an immunoconjugate of formula (II):

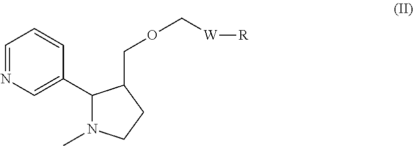

wherein W is a functional group or linker moiety that facilitates linkage to a carrier moiety, and R is a carrier moiety. W may be selected from —H, —OH, ═CH₂, —CH₃, —OCH₃, —COOH, halogen, acyl, activated acyls, such as 2-nitro-4-sulfobenzoate and N-oxysuccinimidate, alkyl, N-maleimides, imino acylate, isocyanates, isothiocyanates, haloformate, vinylsulfone, imidoester, phenylglyoxalate, hydrazide, alkynyl, azido, amino, N-hydroxysuccinimidate, —O—, —(CH₂)ₘ—(wherein m is an integer from about 1 to about 20), —C(O)—, —S—S—, —NH—, —C(O)O—, —C(O)NH—, —N═N—, —N═N═N—, —CH═CH— and —C≡C—. Other suitable linkers of sufficient length and flexibility may also be used with the present invention. A wide range of reagents and/or active groups may be used to facilitate cross-linking of a hapten to a carrier moiety.

In some preferred embodiments, the linker group W is covalently linked to the carrier moiety R via a covalent bond. In some of these embodiments, the covalent bond is not a thioether linkage. When a covalent bond is formed between the linker moiety and the carrier moiety, the linker moiety W present in the immunoconjugate of formula (II) is an activated moiety corresponding to the linker moiety X described above. Thus, the linker moiety W in the immunoconjugate can be
—OY—,
—OCH₂—,
—OCO(CH₂)ₙCOY—,
—OCO(CH₂)ₙCNY—,
—OCO(CH₂)ₙY—,
—OCOCH═Y—,
—OCOCH(O)CH₂—,
—OCOCH(OH)CH₂Y—,
—OCO(CH₂)ₙCH(OH)CH₂Y—,
—OCO(CH₂)ₙCH(O)CH₂Y—,
—OCOC₆H₅—,
—O(CH₂)ₙY—,
—CO₂Y—,
—COY—,
—CO(CH₂)ₙCOY—,
—CO(CH₂)ₙCNY—,
—CONH(CH₂)ₙY—,
—CH₂OCO(CH₂)ₙCOY—,
—CH₂OCO(CH₂)ₙCNY—,
—(CH₂)ₙY—,
—CH₂Z(CH₂)ₙY—,
—(CH₂)ₙ—C₆H₁₀—(CH₂)ₘ—COY—,
—(CH₂)ₙ—C₆H₄—(CH₂)ₘ—COY—,
—NH(CH₂)ₙCOY—,
—NH(CH₂)ₖC₆H₁₀—(CH₂)ₘCOY—,
—NH(CH₂)ₘC₆H₄—(CH₂)ₚCOY—,
—NHCO(CH₂)ₙCOY—,
—NHCO(CH₂)ₖC₆H₁₀—(CH₂)ₘCOY—,
—NHCO(CH₂)ₘC₆H₄—(CH₂)ₚCOY—,
—C≡C—(CH₂)ₙNHY—,
—C≡C—(CH₂)ₙCOY—,
—C≡C—(CH₂)ₙC₆H₁₀—(CH₂)ₘCOY—,
—C≡C—(CH₂)ₙC₆H₁₀—(CH₂)ₘNHY—,
—C≡C—(CH₂)ₘC₆H₄—(CH₂)ₚCOY—,
—C≡C—(CH₂)ₘC₆H₄—(CH₂)ₚNHY—,
—CH═CH—(CH₂)ₙNHY—,
—CH═CH—(CH₂)ₙCOY—,
—CH═CH—(CH₂)ₙC₆H₁₀—(CH₂)ₘCOY—,
—CH═CH—(CH₂)ₙC₆H₁₀—(CH₂)ₘNHY—,
—CH═CH—CH₂)ₘC₆H₄—(CH₂)ₚCOY—,
—CH═CH—(CH₂)ₘC₆H₄—(CH₂)ₚNHY—,
—SCO(CH₂)ₙCOY—,
—S(CH2)ₙY—,
—(CH₂)ₙ—R₁—(CH₂)ᵣ—R₂—Y—,
—Z(CH₂)ₙY—,
—ZCO(CH₂)ₙCOY—
   wherein n is an integer from about 0 to about 20; m is an integer from about 0 to about 6; k is an integer from about 0 to about 20; p is an integer from about 0 to about 6; r is an integer from about 1 to about 20; Z is selected from the group consisting of —O—, —CH₂—, and —NH—; R₁ and R₂ are independently selected from the group consisting of —NHCO—, —CONH—, —CONHNH—, —NHNHCO—, —NHCONH—, —CONHNHCO—, and —S—S—; and Y is selected from the group consisting of —O—, ═CH—, —CH₂—, —CH≡CH—, —OCH₂—, —C(O)—, —C(O)O—, —NH—, —C(O)NH—, —N═N—, —N═N═N—, —S—S—, halogen, acyl, 2-nitro-4-sulfobenzoate, N-oxysuccinimididate, N-maleimides, imino acylate, isocyanates, isothiocyanates, haloformate, vinylsulfone, imidoester, phenylglyoxalate, hydrazide, azido, amino, and N-hydroxysuccinimidate.

Some immunoconjugate of the invention have the structure shown in formula III below:

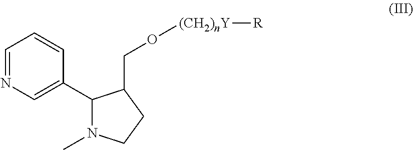

wherein Y is a functional group that facilitates linkage to a carrier moiety, R is a carrier moiety and n is an integer from about 1 to about 20, preferably from about 3 to about 8. Any carrier moiety described above or that is known in the art for conferring immunogenicity to haptens may be used in these immunoconjugates. The linkage between the nicotine hapten and the carrier moiety can be either covalent or non-covalent. In some preferred embodiments, the linkage between the functional group Y and the carrier moiety R is a covalent bond. In some of these embodiments, the covalent bond is not a thioether bond.

In some preferred embodiments, the immunoconjugates of the invention have the structure shown in formula IV below:

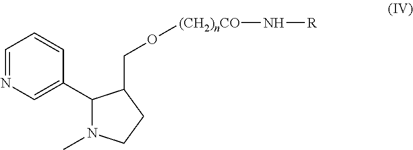

wherein n is an integer from about 1 to about 20, preferably from about 3 to about 8, and R is a carrier protein. In these embodiments, the nicotine hapten is covalently conjugated via an amide bond to the carrier protein. In some more preferred embodiments, n in formula IV is 5, and the carrier protein is TT, CRM or KLH.

Methods of producing the immunoconjugates described herein are also encompassed by the present invention. As noted above, depending on the specific nicotine hapten and carrier moiety, various means can be employed to synthesize an immunoconjugate of the invention. In some preferred embodiments, the invention provides methods of preparing the immunoconjugates of formula III. Typically, the methods entail first converting a compound of formula A to a compound of formula B shown below:

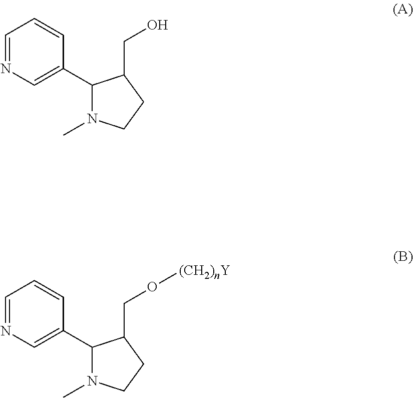

In this step, compound A is derivatized with a functional group Y which can be reactive with a carrier moiety R. To synthesize the immunoconjugate, the functional group Y in compound B is then activated. The hapten compound can thereafter be further converted to the immunoconjugate of formula III by reacting the activated functional group Y with the carrier moiety. As described above, various linker groups can be used to generate the immunoconjugates of the invention. Accordingly, the functional group Y used to activate compound A can be any of the reactive group present in the linker moieties disclosed herein.

Derivatization of compound A with a linker containing a functional group and its further conjugation to a carrier moiety can be carried out via standard chemical reactions or synthesis methods disclosed herein. For example, as demonstrated in the Examples below, compound A can be derivatized by the attachment of a brominated linker to provide a carboxylic acid group. The carboxylic acid group in compound B is then activated, e.g., with 1-ethyl-3-(3-dimethylaminopropyl)carbodiimide (EDC) and sulfo-N-hydroxysuccinimide (S—NHS). The activated hapten compound can thereafter further react with a carrier moiety (e.g., a carrier protein) to generate the immunoconjugates.

### IV. Vaccines Comprising Nicotine Immunoconjugates and their Use for Immunotherapy

The immunoconjugates of the invention can be used to prepare vaccines that are suitable for active immunization protocols. Compositions including the immunoconjugates of the invention can be formulated for in vivo use, e.g., therapeutic or prophylactic administration to a subject. In some particular embodiments, the immunoconjugates are formulated as vaccine compositions.

The preparation of vaccines which contain immunoconjugates as active ingredients is generally well understood in the art. Typically, such vaccines are prepared as injectables, either as liquid solutions or suspensions. Solid forms suitable for formulation in solution or suspension prior to injection may also be prepared. The preparation may also be emulsified. The immunoconjugate may be mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines, as described below. In some pharmaceutical compositions (e.g., vaccines) containing the immunoconjugate of the invention, the intended immune response is enhanced by the inclusion of an adjuvant substance. Adjuvants and their use are well known in the art. Examples of adjuvants include inorganic adjuvants such as aluminium salts (e.g., aluminum phosphate and aluminum hydroxide), organic adjuvants such as Squalene, oil-based adjuvants, and virosomes which contain a membrane-bound hemagglutinin and neuraminidase derived from the influenza virus. Various methods of achieving an adjuvant effect are also known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are incorporated by reference herein.

Vaccines can be conventionally administered to subjects. Preferably, they are administered parenterally by injection, for example, subcutaneously, intracutaneously, intradermally, subdermally or intramuscularly, or any other routes that are suitable for the present invention. Additional formulations which may be suitable for other modes of administration include suppositories and, in some cases, oral, buccal, sublingual, intraperitoneal, intravaginal, epidural, spinal, and intracranial formulations.

As can be appreciated, compositions of the invention should be administered in a manner compatible with the dosage formulation, and in such amount as will be prophylactically or therapeutically effective and immunogenic. The quantity to be administered depends on the subject to be treated, including, e.g., the capacity of the individual's immune system to mount an immune response, and the degree of protection desired. Suitable dosage ranges are from about 0.1 μg/kg body weight to about 10 mg/kg body weight, such as in the range from about 500 μg/kg body weight to about 1000 μg/kg body weight. For example, a dosage range may be from about 0.1 mg/kg body weight, about 0.25 mg/kg body weight, about 0.5 mg/kg body weight, about 0.75 mg/kg body weight, about 1 mg/kg body weight, or about 2 mg/kg body weight, to about 20 mg/kg body weight, about 15 mg/kg body weight, about 10 mg/kg body weight, about 7.5 mg/kg body weight, or about 5 mg/kg body weight. Suitable regimens for initial administration and booster shots are also contemplated and are typified by an initial administration followed by subsequent inoculations or other administrations.

Some embodiments of the invention provide a method of inducing an anti-nicotine immune response in a subject. The subject can be a human or a non-human animal, e.g., a mouse or rat in an animal model. An anti-nicotine immune response specifically refers to induction of a therapeutic or prophylactic nicotine-sequestering effect that is mediated by the immune system of the subject. Such an immune response suitably promotes clearance or immune control of nicotine or nicotine derivatives in the subject. In some embodiments, the anti-nicotine immune response is an antibody response. The antibody response may suitably be the production of IgG, IgA, IgM or IgE antibodies. The anti-nicotine immune response is suitably assessed by methods known in the art, e.g. ELISA for anti-nicotine antibodies. Inducing an anti-nicotine immune response in a subject in accordance with the invention may be accomplished by administering to the subject the immunoconjugate compositions described above.

In some embodiments, the methods of the invention are directed to inducing an anti-nicotine immune response which provides system-wide effects in the subject. The systemic effects can include, e.g., reduction of nicotine withdrawal symptoms, including, but not limited to, craving for cigarettes, irritability, anxiety, restlessness, depressed mood, drowsiness, difficulty concentrating, insomnia, somatic complaints, increased appetite, and weight gain.

### V. Antibodies Specific for Nicotine Haptens

The present invention also provides antibodies that immunoreact with the hapten of this invention. In some embodiments, antibodies of this invention also cross-react with nicotine. In particular embodiments, the antibodies cross-reacts with S-(−), but not R-(+) nicotine. The antibodies may be of any of the immunoglobulin subtypes IgA, IgD, IgG, IgE, or IgM. Antibodies may be produced by any means known in the art and may be, e.g., monoclonal antibodies, polyclonal antibodies, phage display antibodies, and/or human recombinant antibodies. A recombinant antibody can be manipulated or mutated so as to improve its affinity or avidity for the antigen, e.g., a nicotine hapten or nicotine. Means of such manipulation are well known in the art.

In some embodiments, human antibodies or humanized antibodies may be used in passive immunization protocols. Methods to humanize murine monoclonal antibodies via several techniques may be used and are well known in the art. Further, methodologies for selecting antibodies with desired specificity from combinatorial libraries make human monoclonal antibodies directly available. If desired, protein engineering may be utilized to prepare human IgG constructs for clinical applications such as passive immunization of a subject. In passive immunization, a short-term immunization is achieved by the transfer of antibodies to a subject. The antibodies can be administered in a physiologically acceptable vehicle which can be administered by any suitable route, e.g., intravenous (IV) or intramuscular (IM). Any antibodies of the invention described herein may be suitably used, such as monoclonal antibodies (mAb).

The passive administration of anti-nicotine antibodies should prove beneficial to reduce serum levels and attenuate “toxic” (cardiovascular, metabolic, endocrine) effects. It can also be used in weekly or biweekly pharmacotherapy during smoking cessation programs. The pharmacotherapy could entail self-injection of mAb to maintain a high circulating level of antibody. Significantly, in a more user-palatable approach, it may be possible to establish passive mucosal protection against nicotine in the respiratory tract through the use of aerosolized immunoglobulin (see, e.g., Crowe et al., Proc. Natl. Acad. Sci. USA 91:1386-1390, 1994). This method would be particularly applicable to the nicotine dependence problem since the vast majority of users obtain nicotine by smoking.

In some embodiments, active immunization (immunoconjugate vaccine) and passive immunization (antibodies) may be used in combination in a subject. The effective dose of either the immunoconjugate vaccine or antibodies may be the effective dose of either when administered alone. In some embodiments, the effective dose of either in combination with the other may be less than the amount that would be therapeutically effective if either is administered alone.

Some embodiments of the invention provide a method of reducing withdrawal symptoms of nicotine in a subject. Reducing withdrawal symptoms of nicotine can encompass, but is not limited to, reducing craving for cigarettes, irritability, anxiety, restlessness, depressed mood, drowsiness, difficulty concentrating, insomnia, somatic complaints, increased appetite, or weight gain in the subject. Methods of reducing withdrawal symptoms may be accomplished by administering to the subject the immunoconjugate compositions described above in combination with passive immunization or other adjunct therapies used in smoking cessation.

It will be appreciated that the specific dosage of immunoconjugate or antibodies administered in any given case will be adjusted in accordance with the condition of the subject and other relevant medical factors that may modify the activity of the immunoconjugate or antibody or the response of the subject, as is well known by those skilled in the art. For example, the specific dose for a particular patient depends on age, body weight, general state of health, diet, the timing and mode of administration, the rate of excretion and medicaments used in combination. Dosages for a given patient can be determined using conventional considerations such as by means of an appropriate conventional pharmacological protocol.

It is specifically contemplated that any embodiment of any method or composition of the invention may be used with any other method or composition of the invention. As used in this specification and the appended claims, the singular forms “a,” “an,” and “the” include plural referents unless the content clearly dictates otherwise. Thus, for example, reference to a composition containing “an antibody” includes a mixture of two or more antibodies. It should also be noted that the term “or” is generally employed in its sense including “and/or” unless the content clearly dictates otherwise. It also is specifically understood that any numerical value recited herein includes all values from the lower value to the upper value, i.e., all possible combinations of numerical values between the lowest value and the highest value enumerated are to be considered to be expressly stated in this application. For example, if a range is stated as 1% to 50%, it is intended that values such as 2% to 40%, 10% to 30%, or 1% to 3%, etc., are expressly enumerated in this specification.

### EXAMPLES

The following examples are provided to further illustrate the invention but not to limit its scope.

### Example 1

### Materials, Compound Syntheses and Protocols

Materials.

Unless otherwise stated, all reactions were performed under an inert atmosphere with dry reagents, solvents, and flame-dried glassware. (−)-Nicotine and (−)-Cotinine were purchased from Sigma-Aldrich (St. Louis, Mo.). Trans-3′-hydroxymethylnicotine was purchased from Toronto Research Chemicals Inc. (North York, ON). All other chemicals were purchased from major suppliers and used without further purification. Compounds were purified by reverse phase preparative high performance liquid chromatography (HPLC) (Grace, Vydac 218TP C₁₈ 10-15 μm). All compounds were characterized using a Bruker 500 MHz NMR instrument and Agilent LC-MS (ESI) mass spectrometer.

Nicotine Haptens.

Racemic NIC nicotine hapten (Scheme 1) was prepared by reaction of nornicotine with the appropriate linker as previously reported₁₁. AM 1 nicotine hapten was synthesized according to scheme 2. Commercially available trans 3′-hydroxymehtynicotine (20 mg, 0.1 mmol) was added to a cooled stirred solution of NaH (8 mg, 0.3 mmol) in dry DMF (0.5 mL). After 30 min, ethyl 6-(methylsulfonyloxy)hexanoate was added neat and the mixture was allowed to stir at room temperature for 10 hours. The mixture was then cooled to 0° C. and quenched with the addition of 1M HCl. The aqueous layer was extracted twice with diethyl ether and subsequently filtered before HPLC purification [A (aqueous phase)=0.1% TFA H₂O, B (organic phase)=0.1% TFA Acetonitrile; λ=254 nm; solvent gradient 1% B to 15% B in 15 min, 15% B to 95% B in 25 min]. Two peaks of interest were obtained; one main peak corresponded to the final product AM1-COOH while a second smaller one corresponded to the protected ester. After removal of acetonitrile under reduced pressure, the pure fractions were freeze dried to yield AM1-COOH as a pale yellow oil (17.42 mg, 54.7% yield). ₁H NMR (500 MHz, CD₃OD) δ 9.18 (s, 1H), 8.94 (d, J=5.5, 1H), 8.83 (d, J=7.3, 1H), 8.11 (m, 1H), 4.61 (d, J=9.7, 1H), 3.96 (d, J=18.4, 1H), 3.53 (m, 2H), 3.42 (m, 1H), 3.35 (m, 3H), 3.07 (s, 1H), 2.85 (s, 2H), 2.47 (m, 1H), 2.27 (dt, J=15.8, 7.3, 2H), 2.13 (m, 1H), 1.52 (dd, J=15.3, 7.6, 2H), 1.40 (m, 2H), 1.19 (dd, J=14.7, 7.1, 2H). ₁₃C NMR (500 MHz, CD₃OD) δ 177.77, 147.43, 146.33, 136.84, 134.37, 128.92, 101.35, 72.42, 71.68, 57.22, 39.60, 35.11, 34.98, 30.58, 27.12, 26.39, 26.09. LC-MS (M+H)+: calcd for C₁₇H₂₆N₂O₃=307.19; found 307.2.

Hapten-Protein Immunoconjugates.

Racemic MC was conjugated to BSA for ELISA microtiter plate coating only. For AM1 hapten; KLH, TT and CRM conjugates were prepared for immunization. AM1 was activated at room temperature for 6 hrs using standard EDC/sulfo-NHS (1.3 eq each) coupling procedure in DMF. After DMF removal under reduced pressure, the residue was dissolved in 0.1M MOPS saline pH=7.2 and the corresponding amount of protein (1 mg hapten:1 mg protein) was added and allowed to stand for 12 hrs at 4° C. We found MOPS buffer prevented protein unfolding better than PBS. Coupling efficiencies were monitored using MALDI-TOF MS, save for KLH which cannot be directly analyzed. As the number of lysine residues directly affects coupling, TT generally afforded a greater number of hapten copies in line with its higher molecular weight.

Active Immunization Protocols for Mice Studies.

Groups of n=4 129GIₓ mice (6-8 weeks, 23-28 g) were immunized i.p. on days 0, 7, and 133 with a suspension of AM1-TT, AM1-KLH or AM1-CRM (0.1 mg) in phosphate buffered saline (PBS) in formulation with AS-03 adjuvant (GlaxoSmithKline®). On day 7, 14 and 140, serum (0.1 mL) was collected via retroorbital puncture and titers were measured by ELISA. All biological samples collected were stored at −80° C. until use to preserve integrity.

Vaccination of Rats for Self-Administration.

Based on its performance in murine experiments, AM1-TT was advanced onto rat behavioral studies. Wistar-derived male rats (n=5-6, 250-300 g) were purchased from Harlan (Indiana, USA) and assigned either to AM1-TT vaccine or TT-only control group. Rats were immunized with 0.1 mg of immunoconjugate in formulation with AS-03 adjuvant administered into 3 sites (2 s.c.; 1 i.p.). Four total immunizations were performed during the course of the study at days 0, 14, 28 and 53. On days 27, 41 and 72 roughly 0.05 mL of serum was collected onto heparnized microcentrifuge tubes and their immune response to date was measured by ELISA.

Immunologic Assays.

Production of nicotine-specific IgG was measured by ELISA using a NIC-BSA conjugate as the coating antigen. Titers were calculated from the plot of absorbance versus log dilution, as the dilution corresponding to an absorbance reading 50% of the maximal value. NIC-BSA was the antigen of choice in order to prevent biasing of the titer measurements towards the immunized hapten. We have previously demonstrated the suitability of using NIC-BSA for titer measurement and determination of nicotine binding constants.₁₂ NIC-BSA and protein only controls were added to COSTAR 3690 microtiter plates and allowed to dry at 37° C. overnight. Following methanol fixation, non-specific binding was blocked with a solution of 5% non-fat powdered milk in PBS for 0.5 h at 37° C. Next, mouse sera was serially diluted in a 1% BSA solution across the plate and allowed to incubate for 1-2 hrs at 37° C. in a moist chamber. Plates were then washed with DI H₂O and treated with goat antimouse-HRP antibody for 0.5 hr at 37° C. Following another wash cycle, plates were developed with the TMB 2-step kit (Pierce; Rockford, Ill.). In the case of the rat self-administration sera, the absolute titer value obtained is deemed to be “masked” due to concurrent administration of nicotine.

Antibody affinity for nicotine and cotinine, a nicotine major metabolite, was measured by competition ELISA. The same procedure as above was followed except, the desired competitor (nicotine or cotinine) was added concurrently with the mouse sera previous to plate incubation.

Additionally, refined values of antibody affinity and nicotine binding capacity were determined for our rat behavioral study samples via a soluble radioimmunoassay (RIA). A modified version of Muller's method (Muller et al., Meth. Enzymol. 92: 589-601, 1983) was followed as it allows for determination of both affinity constant and concentration of specific antibody in serum. The RIA was carried out in a 96-Well Equilibrium Dialyzer MWCO 5000 Da (Harvard Apparatus, Holliston, Mass.) to allow easy separation of bound and free L-[N-methyl-³H]-Nicotine tracer; specific activity=81.7 Ci/mmol (PerkinElmer, Boston, Mass.). Briefly, rat sera was diluted in MA buffer (sterile filtered 2% BSA in 1×PBS pH=7.4) to a concentration that would bind 40% of ˜24 000 decays/min of 3H-nicotine tracer. A 50 μL aliquot of sera was combined with 10 μL of radiolabelled tracer (˜24 000 decays/min) and 50 μL of unlabeled (−)-nicotine at varying concentrations in RIA buffer; 110 μL of PBS pH=7.4 was added to the solvent chamber and the samples were allowed to reach equilibrium on a plate rotator (Harvard Apparatus, Holliston, Mass.) at room temperature for at least 22 hours. A 70 μL aliquot from each sample/solvent chamber was slowly aspirated and suspended in 5 mL scintillation fluid (Ecolite, ICN, Irvine, Calif.) and the radioactivity of each sample was determined by liquid scintillation spectrometry. These samples were concurrently used to construct a standard curve for use in quantitative ELISA of rat serum samples.

Nicotine Self-Administration.

As stated, the most promising immunoconjugate was moved forward into a rat behavioral model. Wistar-derived male rats (n=5-6, 250-300 g) were purchased from Harlan (Indiana, USA) and were housed in groups of two and maintained in a temperature controlled environment on a 12 h:12 h light cycle. Upon arrival to the laboratory, animals were given free access to food and water during a one-week habituation period. All animal care and use was performed according to NIH guidelines and in compliance with protocols approved by the Institutional Animal Care and Use Committee. Food training and nicotine self-administration took place in single-lever standard Coulbourn operant chambers housed in a sound-attenuated box. Each set of rats received a 5-day long food training session to establish lever pressing prior to self-administration. Initially, rats were restricted to 15 g of food daily (˜85% of free feeding body weight). After the second day of food restriction, rats were trained to respond for food under a fixed ratio 1 (FR1) schedule of reinforcement (i.e. 1 food pellet per lever press) with a 1-second time out (TO-1 s). Training sessions lasted 30 min daily and once a steady baseline was established, rats were returned to ad libitum food in preparation for intravenous jugular catheter implant surgery.

Upon successful completion of surgery, rats were allowed to recover for 3-5 days before starting the self-administration sessions. During the recovery period, rats remained on ad libitum food access and had catheter lines flushed daily to prevent blood coagulation and infection. Intravenous infusions (0.1 mL) are delivered over one second interval via infusion pump (Razel, CT). Following successful recovery, rats were again food deprived in preparation of nicotine self-administration sessions. Subjects were trained to intravenously self-administer nicotine at a dose of 0.03 mg/kg/infusion during 1-hour self-administration sessions, 5 days/week under a FR1-TO-20 s schedule until stable responding was achieved. Stable responding was defined as less than 20% variability across 3 consecutive sessions. Following establishing of baseline, the test vaccine was administered as described.

In between vaccinations, rats were tested either on a FR-1-TO-20 s or progressive ratio (PR) schedule of reinforcement during 1-hr sessions for 3-4 days/week. Rats were flushed with saline before the beginning of each session in order to ensure catheter patency, and after each session again received saline and Timentin to prevent blood coagulation and infection. Rats that lost catheter patency were subsequently removed from the experiment. Data was collected online simultaneously from multiple operant chambers. Results of the operant procedure are reported as mean cumulative number of bar presses for nicotine.

### Example 2

### Synthesis of Nicotine Hapten AM1 and Immunoconjugates

AM1 is an unconstrained hapten that follows the 3′-substituted general structure as shown in FIG. 1. AM1 possesses a hapten design advantageous over those set forth by Langone and Pentel (Langone et al., Biochemistry 12:5025-5030, 1973; and Hieda et al., Int J Immunopharmacol 22:809-819, 2000). The presence of a liable ester linkage in the Langone design translates into spontaneous detachment of antigen from the carrier protein and thus loss of anti-nicotine immunogenicity; while such a design would not preclude monoclonal antibody production it could severely impinge on an active vaccine's efficacy. Spontaneous ester hydrolysis under physiological conditions is a known phenomenon and increasing hapten stability via an ester-amide interchange was previously successfully investigated for cocaine immunopharmacotherapeutical efforts (Carrera et al., Proc Natl Acad Sci 98; 1988-1992, 2001). This could explain why Pentel made said modification to the Langone design (Hieda et al., Int J Immunopharmacol 22:809-819, 2000). Amide bonds are resistant to hydrolysis unless specific proteases are present which significantly minimizes the possibility of loss of hapten cargo during immunization. However, while a peptide bond is able to confer hapten stability, it also introduces an additional immunogenic moiety to the target structure.

Detailed steps of synthesizing the nicotine haptens are described above. In the case of AM1, we have substituted the amide moiety with a simple ether appendage which not only provides hapten stability but also allows for a “masked” appendage site which focuses the immune response onto the desired nicotinic target. The ether linkage effectively mimics a lipid structure to have prominent non-immunogenic character and low cytotoxicity. It thus allows for a muted linker attachment site from an immunological standpoint.

Upon confirmation of hapten design, we set out to generate the proposed structure. Initial synthetic efforts for AM1 consisted of reacting commercially available 3′-hydroxymethylnicotine with a brominated linker; a reaction that proceeded in low yields despite multiple attempts to optimize the conditions with addition of AgO₂ or tetrabutylammonium iodide (TBAI). Substitution of the bromine moiety with a more electrophilic mesylate leaving group finally afforded the desired product in 54% yield (Scheme 1).

In an effort to elucidate the optimal carrier vehicle for immunization, AM1 was conjugated with three different carrier proteins, keyhole limpet hemocyanin (KLH), tetanus toxoid (TT), and diphtheria toxin cross-reactive mutant 197 (CRM). Each protein was chosen based on its ability to elucidate a potent immune response. Coupling was achieved using a two step heteroligation technique. Hapten activation with 1-ethyl-3-(3-dimethylaminopropyl)carbodiimide (EDC) and sulfo-N-hydroxysuccinimide (S-NHS) followed by addition of the carrier protein promoted attack of the lysine residues onto the activated carboxylic acid to form the desired hapten-protein conjugates (Scheme 1). Coupling efficiencies were monitored using MALDI mass spectrometry, save for KLH which cannot be directly analyzed using MS. It is clear that the number of lysines on each protein directly determines the number of hapten copies that may be attached, thus TT generally afforded a greater number of hapten molecules than CRM in line with its higher molecular weight.

### Example 3

### Immunogenicity of Nicotine Hapten AM1 Based Immunoconjugates

This Example describes immunogenicity of nicotine hapten immunoconjugates and antibodies generated in mice. In an effort to elucidate the optimal carrier vehicle for immunization, AM1 hapten was conjugated with three different carrier proteins, namely KLH, TT and CRM. The efficacy of all AM1 hapten-protein immunoconjugates was assessed by vaccination into 129GIₓ mice using standard immunization protocols. Specifically, 100 μg of the hapten conjugate were mixed with the desired adjuvant and immediately injected into mice (i.p., n=4 per hapten conjugate group). Three test groups were included, AM1-KLH, AM1-TT and AM1-CRM, plus three carrier only controls. In all cases the vaccine was formulated with AS-03, an emulsion based proprietary adjuvant from GlaxoSmithKline. The immunization schedule was set as follows: injections were done at t=0, t=14 d and t=133 d while bleeds were taken a week after each injection namely at t=7 d, t=21 d and t=140 d. In all cases the immunoconjugates showed no toxicity or deviation from the norm in mice. All biological samples collected were stored at −80° C. until use to preserve their integrity.

Antibody titers are critical to the preclinical evaluation of any vaccine candidate as they directly measure the immunogenicity of a given hapten. An enzyme-linked immunosorbent assay (ELISA) was used to assess the magnitude (titer), as well as the average affinity (K_{d}) and specificity of the antibodies generated during vaccination. In order to prevent biasing of the titer measurement results during the ELISA screening, a non-immunized antigen, namely NIC-BSA, was used for coating of the microtiter plates. We have previously demonstrated the suitability of using NIC-BSA immunoconjugate for titer measurements and determination of nicotine binding constants (Meijler et al., J Am Chem Soc 125:7164-7165, 2003). Our reasoning here being that this hapten closely resembles free nicotine in solution as its molecular structure is simply that of the native nicotinic nucleus with a linkage through the pyrrolidine N-methyl group. In our previous studies enantiomerically pure NIC-BSA was utilized, however, as production of this hapten is synthetically demanding and expensive we have prepared the racemic version of this hapten in one step from nornicotine and our previously reported β-alanine linker. It was found that racemic NIC-BSA is equally effective as its enantiomerically pure counterpart (data not shown).

TABLE 1
Average titer and nicotine binding constant measurements obtained
from n = 4 immunized 129GI^{x} mice on NIC-BSA coated plates.

| | | AS03 | | | |
|---|---|---|---|---|---|
| | | 2nd bleed | | 3rd bleed | |
| | | Titer | Kd (μM) | Titer | Kd (μM) |
| AM1 | KLH | 18133 | 44.16 ± 4.61 | 21333 | 27.39 ± 8.69 |
| | TT | 28000 | 102.94 ± 11.40 | 94400 | 14.63 ± 2.19 |
| | CRM | 22400 | 138.16 ± 17.72 | 64000 | 13.22 ± 1.54 |

Bleeds obtained from test groups after only one injection at t=7 d showed no significant titer and thus were deemed inadequate for any further analysis. Similar results were obtained for all bleeds from the control protein-only group. It is well established in the literature that multiple immunizations are required for adequate stimulation even with the most successful vaccines (Lu, Curr Op Immunol 21:346-351, 2009). Additional challenges with extra injections are expected to give a more robust response as it increases the interaction time between antigen and immune system. Thus while a significant response was observed after two injections, the effect of a third injection following ˜4 months of “rest” was also assessed. Results obtained are summarized in Table 1. While an increase in titer was observed with all three test groups, the most drastic improvement in immunogenicity was seen with AM1-TT where the titer measurement increased more than 3-fold after a third injection to reach ˜1:100,000 titer levels.

While antibody titer data can be extremely promising and reveal the overall immunogenicity of a vaccine candidate, an equally important parameter in predicting efficacy is the ability of the polyclonal antibody response to bind its desired antigen, in this case, nicotine. It is important to note that the binding constant measured via competition ELISA are inherently average constants and thus are higher than what would be observed with other more precise methods such as equilibrium dialysis with radioactive labeled drug (Meijler et al., J Am Chem Soc 125:7164-7165, 2003). Due to the ease of analysis, we used competition ELISA as a first line of analysis for our purposes. We expect a viable active vaccine candidate to show binding constants in the order of 10 μM versus the sub-μM to nM constants typically observed with monoclonal antibodies. For our analysis, a measurable titer against NIC-BSA must be present in order to measure the nicotine binding constant as this is the relevant competition we are attempting to measure (i.e. NIC-BSA versus free nicotine in solution). As stated, no competition data is available for the first bleed as well as the control groups. The second bleed data shows some affinity towards nicotine but acceptable levels were not achieved until the third injection. In particular the TT and CRM groups appear to greatly benefit from an additional challenge as the affinity towards nicotine is increased by a full order of magnitude between the two data points. Importantly, this was accomplished without losing specificity as binding constants against cotinine, nicotine's main metabolite, remained negligible in line with what was reported with similar haptens by Hieda et al., Int J Immunopharmacol 22:809-819, 2000.

### Example 4

### Behavioral Effects of Nicotine Hapten AM1 Based Immunoconjugates

This Example describes assessment of behavioral changes induced by immunization of AM1 based immunoconjugates on rats trained to intravenously self-administer nicotine. Based on their performance in murine experiments, AM1-TT hapten-protein conjugate was advanced to such behavioral studies. Rats have a well-characterized central nervous system whose neurochemical pathways, particularly in the limbic and motivational parts of the brain, correspond qualitatively to that of humans. Their behavioral repertoire is well characterized and shows a characteristic dependence syndrome during chronic administration.

The rats were trained to intravenously self-administer nicotine, at a dose of 0.03 mg/kg/infusion during 1-hour sessions. The objective of the self-administration experiment was to assess any behavioral changes induced by vaccination on rats. This dose was used to mimic the intake of a heavy smoker as 0.03 mg/kg per hour is roughly equal to the nicotine infusion of 2 cigarettes in a human (Hieda et al., 2000). Two groups were included in the study, a TT-protein only control group (n=6) and AM1-TT (n=5). Cell sizes of n=5-6 were considered enough to provide reliable estimates of drug effects. Wistar-derived male rats (250-300 g) were housed in groups of two and maintained in a temperature controlled environment on a 12 h:12 h light cycle. Upon arrival to the laboratory, animals were given free access to food and water during a one-week habituation period. Animals used in this study were handled, housed and sacrificed in accord with the current NIH guidelines regarding the use and care of laboratory animals, and all applicable local, state, and federal regulations and guidelines.

Food training and nicotine self-administration took place in standard Coulbourn operant chambers housed in a sound-attenuated box. Operant chambers are equipped with a single lever, mounted 2-cm above the floor, and a cue light mounted 2-cm above the lever on the back wall of the chamber. For food training, a food hopper was located to the left of the lever, in the middle of the back wall. Rats were manipulated daily for several days prior to experimental testing in order to desensitize them to handling stress. Each set of rats, then received a 5 day long food training session to establish lever pressing prior to drug self-administration. Initially, rats were restricted to 15 grams of food daily (equivalent to ˜85% of their free-feeding body weight). After the second day of food restriction, rats were trained to respond for food under a fixed ration 1 (FR1) schedule of reinforcement (i.e. 1 food pellet per lever press) with a 1-second time out (TO-1 s). Training sessions lasted 30 min daily and once rats obtained steady baseline responding to a FR1-TO-20 s schedule, they were returned to ad libitum food in preparation for intravenous jugular catheter implant surgery.

Upon successful completion of surgery, rats were allowed to recover for 3-5 days before starting the self-administration sessions. During the recovery period, rats remained on ad libitum food access and had catheter lines flushed daily to prevent blood coagulation and infection. Intravenous infusions are delivered in a volume of 0.1 mL over a one second interval, via an infusion pump (Razel, CT). Following successful recovery, rats were again food deprived to 85% of their free-feeding body weight. Once self-administration sessions began, subjects were trained to intravenously self-administer nicotine at a dose of 0.03 mg/kg/infusion, during 1-hour self-administration sessions, 5 days/week under an FR1-TO-20 s schedule of reinforcement until stable responding was achieved. Stable responding was defined as less than 20% variability across 3 consecutive sessions. After stable responding was achieved, the test vaccine was administered according to standard immunization procedures. Namely, 100 μg of immunoconjugate was mixed with emulsion based AS-03 adjuvant and administered into 3 sites; two s.c. and one i.p. AM1-TT immunoconjugates were produced as described above and good coupling efficacy was observed with 21-22 copies of hapten present. A total of 4 injections were administered as follows: t=0, t=14 d, t=28 d, and t=53 d. Rats were bled onto heparnized microcentrifuge tubes roughly two weeks after each injection as follows: t=27 d, t=41 d and t=72 d.

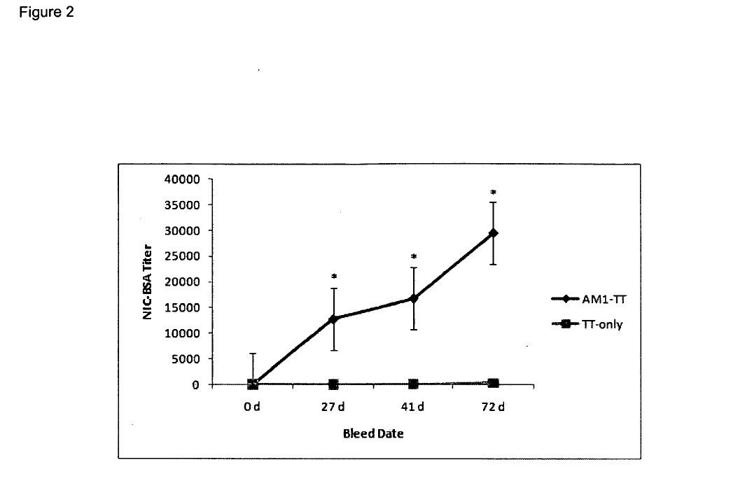

Sera collected during the course of self-administration were tested for presence of nicotine specific antibodies on NIC-BSA coated microtiter plates. Importantly, and as expected, all samples from the TT-only immunized controls showed no titers on NIC-BSA. Samples collected from the AM1-TT vaccinated groups demonstrated a steady increase in titer over time after each boost and the maximum average level achieved was 1:30,000 for the last bleed (FIG. 2). At the time of the last bleed, K_{d} for nicotine was 5.68±0.80 nM as calculated by soluble RIA. Nicotine binding capacity calculated from these data was 5.36±1.20×10⁻⁷ M, which is equivalent to 40.26±8.97 μg/mL of nicotine-specific IgG. IgG was assumed to have a molecular weight of 150 kDa and two nicotine-binding sites per molecule. This nicotine specific IgG concentration in serum corresponds to a nicotine binding capacity in serum of 87.10±19.40 ng/mL.

Importantly, at the time of the third bleed we do not observe drastic variation in titers. The difference between the high and low responder was 4-fold (min. titer value 1:12,800; max. titer value 1:51, 200), as opposed to 10-fold reported in, e.g., Hieda et al., Int J Immunopharmacol 22:809-819, 2000. The titer median value was 1:25,600. Statistical significance was determined by using a two tailed student t-test. These data support our hypothesis of advantageous hapten design.

The binding constants measured showed medium affinity to nicotine, moderately higher than what was observed in mice. The average nicotine binding constant for the third bleed was 66.04±34.19 μM (vs ˜15 μM in mice). Nonetheless, the antibodies elucidated retained good specificity against nicotine and were unable to bind cotinine, a major nicotine metabolite.

We then examined behavior effects of nicotine self-administration in rats. In between vaccinations, rats were tested either on a FR-1-TO-20 s fixed ratio or progressive ratio of reinforcement during 1-hr sessions for 3-4 day/week. Rats were flushed with saline before the beginning of each session in order to ensure catheter patency, and after each session again received saline and Timentin to prevent blood coagulation and infection in the catheters. Rats with catheters no longer patent were subsequently removed from the experiment. Data was collected on-line simultaneously from multiple operant chambers. Results of the operant procedure are reported as mean cumulative number of bar presses for nicotine. TT immunized rats represent the low to no-NIC-BSA titer group while AM1-TT rats correspond to moderate to high titer subjects.

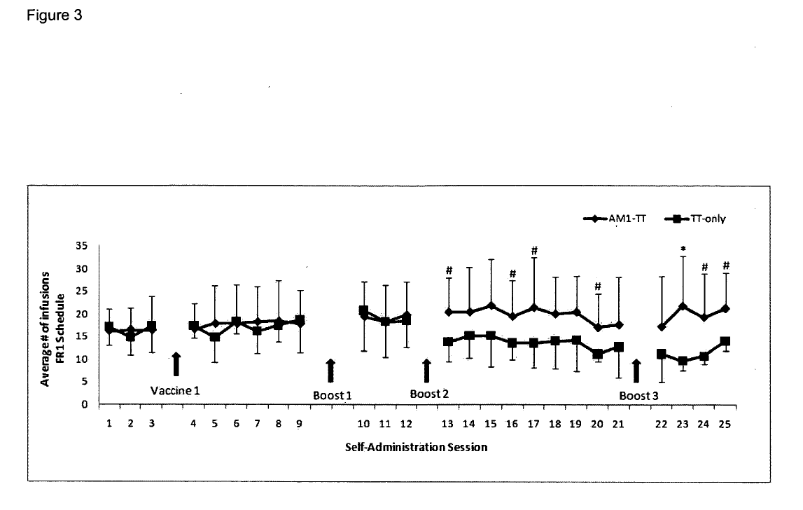

It is well documented that in the absence of high titers, the protective effects of vaccination are not reflected in behavioral changes (Carrera et al., Proc Natl Acad Sci 97:6202-6206, 2000). Despite failing to obtain titers or affinity as high as what was observed in mice, vaccination of rat subjects with AM1-TT immunoconjugate resulted in a separation of the self-administration patterns observed between groups (FIG. 3). At the later self-administration sessions, the AM1-TT immunized group showed a moderate increase in the number of nicotine lever presses. Based on titer information, we predicted a change between the two groups and thus deemed a one-tailed student t-test to be appropriate to calculate the statistical significance of this change. The results shown in FIG. 3 indicate that the behavioral effects observed are moderately significant (average p value for sessions 22-25=0.09). We believe this separation is the result of the steady increase in NIC-BSA titers following additional booster injections. Also, higher levels of statistical significance (p<0.05) for this separation could be reached if a larger sample set is used or if a longer vaccination schedule is used.

One likely interpretation of these results is that the rats responded by modifying their SA behavior and attempted to surmount these protective effects via higher drug intake. AM1-TT immunized animals were effectively “working-harder” to get the nicotine induced rewarding effects. Similar results were obtained during cocaine self-administration experiments which uncovered an inverted U-shaped function shift during competitive drug antagonism (Carrera et al., Proc Natl Acad Sci 97:6202-6206, 2000). That is, lower doses of cocaine will be self-administered similar to the level of saline (non-reinforcer) and higher doses will be more self-administered with a shorter interval between injections. It is common to observe that animals increase the rate of responding under an FR schedule when the unit dose of cocaine for self-administration decreases which suggests that the animals compensate the decreased unit amount of cocaine per injection by increasing the rate of injection. Therefore, increased responding for cocaine under an FR schedule by immunization may suggest that rats compensate the partial blockade of cocaine delivery to the CNS in the presence of antibodies by increasing the rate of cocaine self-administration. Translated to the human nicotine addiction, immunized subjects would find the cost of smoking greatly increased (as if the cost of a pack of cigarettes had been doubled).

Finally, while this study did not attempt to elicit the tolerability of the hapten-protein conjugates, we note that all animals survived immunization and none presented adverse side effects stemming from injection during the course of the experiment. In order to assess the overall health of the test subjects, the body weight of all animals was monitored for the length of the study. We found that AM1-TT immunized animals presented slightly lower weights than control. At t=72 d, AM1-TT immunized groups weighed an average of 10% less than those of the TT-only control group (data not shown).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A hapten of formula (I): 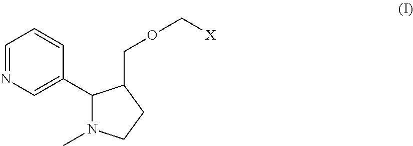
wherein X is a linker moiety that does not contain a thiol group.

2. The hapten of claim 1, wherein X is selected from the group consisting of:
—OY,
—OCH₃,
—OCO(CH₂)ₙCOY,
—OCO(CH₂)ₙCNY,
—OCO(CH₂)ₙY,
—OCOCH═Y,
—OCOCH(O)CH₂,
—OCOCH(OH)CH₂Y,
—OCO(CH₂)ₙCH(OH)CH₂Y,
—OCO(CH₂)ₙCH(O)CH₂Y,
—OCOC₆H₅,
—O(CH₂)ₙY
—CO₂Y,
—COY,
—CO(CH₂)ₙCOY,
—CO(CH₂)ₙCNY,
—CONH(CH₂)ₙY,
—CH₂OCO(CH₂)ₙCOY,
—CH₂OCO(CH₂)ₙCNY,
—(CH₂)ₙY,
—CH₂Z(CH₂)ₙY,
—(CH₂)ₙ—C₆H₁₀—(CH₂)ₘ—COY,
—(CH₂)ₙ—C₆H₄—(CH₂)ₘ—COY,
—NH(CH₂)ₙCOY,
—NH(CH₂)ₖC₆H₁₀—(CH₂)ₘCOY,
—NH(CH₂)ₘC₆H₄—(CH₂)ₚCOY,
—NHCO(CH₂)ₙCOY,
—NHCO(CH₂)ₖC₆H₁₀—(CH₂)ₘCOY,
—NHCO(CH₂)ₘC₆H₄—(CH₂)ₚCOY,
—C≡C—(CH₂)ₙNHY,
—C≡C—(CH₂)ₙCOY,
—C≡C—(CH₂)ₙC₆H₁₀—(CH₂)ₘCOY,
—C≡C—(CH₂)ₙC₆H₁₀—(CH₂)ₘNHY,
—C≡C—(CH₂)ₘC₆H₄—(CH₂)ₚCOY,
—C≡C—(CH₂)ₘC₆H₄—(CH₂)ₚNHY,
—CH═CH—(CH₂)ₙNHY,
—CH═CH—(CH₂)ₙCOY,
—CH═CH—(CH₂)ₙC₆H₁₀—(CH₂)ₘCOY,
—CH═CH—(CH₂)ₙC₆H₁₀—(CH₂)ₘNHY,
—CH═CH—CH₂)ₘC₆H₄—(CH₂)ₚCOY,
—CH═CH—(CH₂)ₘC₆H₄—(CH₂)ₚNHY,
—SCO(CH₂)ₙCOY,
—S(CH2)ₙY,
—(CH₂)ₙ—R₁—(CH₂)ᵣ—R₂—Y,
—Z(CH₂)ₙY,
—ZCO(CH₂)ₙCOY
wherein n is an integer is from about 1 to about 20; m is an integer from about 0 to about 6; k is an integer from about 0 to about 20; p is an integer from about 0 to about 6; r is an integer from about 1 to about 20; Z is selected from the group consisting of —O—, —CH₂—, and —NH—; R₁ and R₂ are independently selected from the group consisting of —NHCO—, —CONH—, —CONHNH—, —NHNHCO—, —NHCONH—, —CONHNHCO—, and —S—S—; and Y is selected from the group consisting of —H, —OH, ═CH₂, —CH₃, —OCH₃, —COOH, halogen, acyl, 2-nitro-4-sulfobenzoate, N-oxysuccinimididate, N-maleimides, imino acylate, isocyanates, isothiocyanates, haloformate, vinylsulfone, imidoester, phenylglyoxalate, hydrazide, azido, amino, and N-hydroxysuccinimidate.

3. The hapten of claim 1, wherein R is —Z(CH₂)ₙY, wherein n is an integer from about 1 to about 20, Z is selected from the group consisting of —N—, —CH₂— and —O—, and Y is selected from the group consisting of —OH, —OCH₃, —COOH, acyl, aryl, alkyl, N-maleimides, imino acylate, isocyanates, isothiocyanates, haloformate, vinylsulfone, imido acylate, phenylglyoxalate, hydrazide, alkynyl, azido, amino, and N-hydroxysuccinimidate.

4. The hapten of claim 3, wherein Z is —CH₂— and n is 3.

5. The hapten of claim 3, wherein Y is —COOH.

6. An immunoconjugate of formula (II): 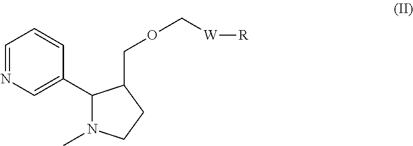
wherein W is a linker moiety that is covalently linked to a carrier moiety R, and wherein the covalent linkage is not a thioether bond.

7. The immunoconjugate of claim 6, wherein W is selected from the group comprising:
—OY—,
—OCH₂—,
—OCO(CH₂)ₙCOY—,
—OCO(CH₂)ₙCNY—,
—OCO(CH₂)ₙY—,
—OCOCH═Y—,
—OCOCH(O)CH₂—,
—OCOCH(OH)CH₂Y—,
—OCO(CH₂)ₙCH(OH)CH₂Y—,
—OCO(CH₂)ₙCH(O)CH₂Y—,
—OCOC₆H₅—,
—O(CH₂)ₙY—
—CO₂Y—,
—COY—,
—CO(CH₂)ₙCOY—,
—CO(CH₂)ₙCNY—,
—CONH(CH₂)ₙY—,
—CH₂OCO(CH₂)ₙCOY—,
—CH₂OCO(CH₂)ₙCNY—,
—(CH₂)ₙY—,
—CH₂Z(CH₂)ₙY—,
—(CH₂)ₙ—C₆H₁₀—(CH₂)ₘ—COY—,
—(CH₂)ₙ—C₆H₄—(CH₂)ₘ—COY—,
—NH(CH₂)ₙCOY—,
—NH(CH₂)ₖC₆H₁₀—(CH₂)ₘCOY—,
—NH(CH₂)ₘC₆H₄—(CH₂)ₚCOY—,
—NHCO(CH₂)ₙCOY—,
—NHCO(CH₂)ₖC₆H₁₀—(CH₂)ₘCOY—,
—NHCO(CH₂)ₘC₆H₄—(CH₂)ₚCOY—,
—C≡C—(CH₂)ₙNHY—,
—C≡C—(CH₂)ₙCOY—,
—C≡C—(CH₂)ₙC₆H₁₀—(CH₂)ₘCOY—,
—C≡C—(CH₂)ₙC₆H₁₀—(CH₂)ₘNHY—,
—C≡C—(CH₂)ₘC₆H₄—(CH₂)ₚCOY—,
—C≡C—(CH₂)ₘC₆H₄—(CH₂)ₚNHY—,
—CH═CH—(CH₂)ₙNHY—,
—CH═CH—(CH₂)ₙCOY—,
—CH═CH—(CH₂)ₙC₆H₁₀—(CH₂)ₘCOY—,
—CH═CH—(CH₂)ₙC₆H₁₀—(CH₂)ₘNHY—,
—CH═CH—CH₂)ₘC₆H₄—(CH₂)ₚCOY—,
—CH═CH—(CH₂)ₘC₆H₄—(CH₂)ₚNHY—,
—SCO(CH₂)ₙCOY—,
—S(CH₂)ₙY—,
—(CH₂)ₙ—R₁—(CH₂)ᵣ—R₂—Y—,
—Z(CH₂)ₙY—,
—ZCO(CH₂)ₙCOY—
wherein n is an integer is from about 1 to about 20; m is an integer from about 0 to about 6; k is an integer from about 0 to about 20; p is an integer from about 0 to about 6; r is an integer from about 1 to about 20; Z is selected from the group consisting of —O—, —CH₂—, and —NH—; R₁ and R₂ are independently selected from the group consisting of —NHCO—, —CONH—, —CONHNH—, —NHNHCO—, —NHCONH—, —CONHNHCO—, and —S—S—; and Y is selected from the group consisting of —O—, ═CH—, —CH₂—, —CH═CH—, —C≡C—, —OCH₂—, —C(O)—, —C(O)O—, —NH—, —C(O)NH—, —N═N—, —N═N═N—, —S—S—, halogen, acyl, 2-nitro-4-sulfobenzoate, N-oxysuccinimididate, N-maleimides, imino acylate, isocyanates, isothiocyanates, haloformate, vinylsulfone, imidoester, phenylglyoxalate, hydrazide, azido, amino, and N-hydroxysuccinimidate.

8. The immunoconjugate of claim 6, wherein W is —Z(CH₂)ₙY, wherein n is an integer from about 1 to about 20, Z is selected from the group consisting of —NH—, —O—, and —CH₂—, and Y is selected from the group consisting of —O—, ═CH—, —CH₂—, —CH═CH—, —C≡C—, —OCH₂—, —C(O)—, —C(O)O—, —NH—, —C(O)NH—, —N═N—, —N═N═N—, —S—S—.

9. The immunoconjugate of claim 8, wherein Z is —CH₂— and n is 3.

10. The immunoconjugate of claim 8, wherein Y is —C(O)O—.

11. The immunoconjugate of claim 6, wherein R is selected from the group comprising keyhole limpet hemocyanin (KLH), edestin, thyroglobulin, human serum albumin, sheep red blood cells (sheep erythrocytes), tetanus toxoid (TT), diphtheria toxoid, cholera toxoid, polyamino acids, D-lysine, D-glutamic acid, members of the LTB family of bacterial toxins, retrovirus nucleoprotein (retro NP), rabies ribonucleoprotein (rabies RNP), vesicular stomatitis virus nucleocapsid protein (VSV-N), recombinant pox virus subunits, and bovine serum albumin (BSA).

12. The immunoconjugate of claim 6, wherein the carrier moiety is tetanus toxoid (TT), diphtheria toxin cross-reactive mutant 197 (CRM), keyhole limpet hemocyanin (KLH) or BSA.

13. A composition comprising an immunologically effective amount of the immunoconjugate of claim 6 and a physiologically acceptable vehicle.

14. The composition of claim 13, further comprising an adjuvant.

15. A method of inducing an anti-nicotine immune response in a subject comprising immunizing the subject with an immunologically effective amount of the composition of claim 13.

16. The method of claim 15, wherein X is —(CH₂)₄—C(O)O—, and the carrier moiety is tetanus toxoid (TT), diphtheria toxin cross-reactive mutant 197 (CRM), keyhole limpet hemocyanin (KLH) or BSA.

17. A method of preparing an immunoconjugate of formula III: 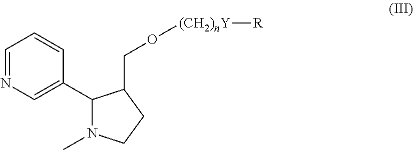
wherein Y is a functional group that facilitates linkage to a carrier moiety, R is a carrier moiety and n is an integer from about 3 to about 8, the method comprising:
(a) converting compound A: 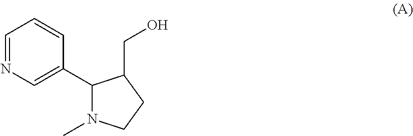
to compound B: 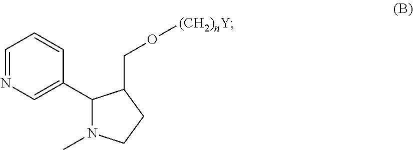
and
(b) converting compound B to the immunoconjugate of formula III.

18. The method of claim 17, wherein n is 5, and Y is —C(O)O—.

19. The method of claim 17, wherein the carrier moiety is tetanus toxoid (TT), diphtheria toxin cross-reactive mutant 197 (CRM), keyhole limpet hemocyanin (KLH) or BSA.

20. An antibody that binds the immunoconjugate of claim 6.

21. The antibody of claim 20, wherein the antibody binds nicotine.

22. The antibody of claim 20, wherein the antibody binds nicotine with a dissociation constant of about 150 μM to about 10 μM.

23. A composition comprising the antibody of claim 20 and a physiologically acceptable vehicle.
